(19) United States
(12) Patent Application Publication
(10) Pub. No.: US 2025/0118438 A1
(43) Pub. Date: Apr. 10, 2025

(54) INFORMATION PROCESSING SYSTEM AND INFORMATION PROCESSING METHOD

(71) Applicant: HAKUHODO DY HOLDINGS INC., Tokyo (JP)

(72) Inventors: Seiichi IGAYA, Tokyo (JP); Hiroshi KATO, Tokyo (JP)

(21) Appl. No.: 18/694,768

(22) PCT Filed: Sep. 16, 2022
(86) PCT No.: PCT/JP2022/034816

§ 371 Date: Mar. 22, 2024

(30) Foreign Application Priority Data
Sep. 30, 2021 (JP) 2021-161424

(51) Int. Cl. G16H 50/30; G16H 50/20

(52) U.S. Cl. CPC G16H 50/30; G16H 50/20

(57) ABSTRACT

According to one aspect of the present disclosure, ability values of first subjects are calculated based on first test data. Each of the ability values is an ability value relating to a motor function of a body of a corresponding one of the first subjects. The first test data describes a test result relating to the motor function of each of the first subjects. Furthermore, a calculation formula for a score corresponding to an ability value is set based on a distribution of the ability values relative to a subject's attribute. Based on an ability value of a second subject relating to the motor function and on the calculation formula, a score corresponding to the ability value of the second subject is calculated.

### CROSS-REFERENCE TO RELATED APPLICATION

This international application claims the benefit of Japanese Patent Application No. 2021-161424 filed on Sep. 30, 2021 with the Japan Patent Office, and the entire disclosure of Japanese Patent Application No. 2021-161424 is incorporated herein by reference.

### TECHNICAL FIELD

The present disclosure relates to an information processing system and an information processing method.

### BACKGROUND ART

There is a known motor function evaluation apparatus for evaluating motor function of subjects (see, for example, Patent Document 1). This apparatus determines a level of locomotive syndrome (syndrome of the musculoskeletal system) using a load sensor, based on a period of time in which the position of the center of gravity of the subject is stable.

Also, there is a known technique of determining a level of locomotive syndrome from tests: a stand-up test for examining lower limb muscle strength, a 2-step test for examining stride length, and a questionnaire test with questions for checking physical conditions and daily life.

### PRIOR ART DOCUMENTS

### Patent Documents

Patent Document 1: Japanese Unexamined Patent Application Publication No. 2018-102594

### SUMMARY OF THE INVENTION

### Problems to be Solved by the Invention

Physical motor function generally decreases with aging. A level of the motor function varies depending on sex. Thus, simply determining the level of locomotive syndrome and indicating the determined level by conventional techniques does not allow users to determine whether their motor function reaches a standard level for attributes such as age and sex.

In one aspect of the present disclosure, it is desirable to provide a technique that can easily determine whether a level of a motor function of a subject is high or low, taking into account attributes of the subject such as age.

### Means for Solving the Problems

According to one aspect of the present disclosure, an information processing system is provided. The information processing system comprises an ability value calculator, a calculation formula setter, a score calculator, and an outputter. The ability value calculator is configured to calculate ability values of first subjects based on first test data. Each of the ability values is an ability value relating to a motor function of a body of a corresponding one of the first subjects. The first test data may describe a test result relating to the motor function of each of the first subjects. The test result may include determination values, for respective test items, for a corresponding one of the first subjects in each of the test items.

The calculation formula setter is configured to set a calculation formula for a score corresponding to an ability value based on a distribution of the ability values calculated by the ability value calculator relative to a subject's attribute. The score calculator is configured to calculate the score corresponding to an ability value of a second subject relating to the motor function based on the ability value of the second subject and on the calculation formula. The second subject may be different from the first subjects or the same as any of the first subjects. The second test data may include determination values, for the respective test items relating to the motor function of the second subject.

The outputter is configured to output the score calculated by the score calculator. The subject's attribute may be defined by an age of a subject. Examples of the subject's attribute include an attribute defined by a combination of a sex and an age of the subject. The calculation formula may be a calculation formula for calculating, from the ability value, a health age relating to the motor function as the score.

According to the information processing system configured to calculate the health age using the aforementioned method, it is possible to easily determine, based on the health age, whether the level of the subject's motor function taking into account the sex and age is high or low. For example, the subject can easily determine, based on the health age that has been informed, whether the level of the motor function taking into account his or her own age is high or low.

According to one aspect of the present disclosure, the calculation formula setter may be configured to set the calculation formula in accordance with a regression model between the age and the ability value, based on a regression analysis using, as samples, the ability values of the first subjects. Use of the regression analysis makes it possible to calculate an appropriate health age from a statistical perspective, and inform the subjects about a more precise level of the motor function.

According to one aspect of the present disclosure, an alternative information processing system may be provided. The alternative information processing system comprises an ability value calculator configured to calculate ability values of two or more first subjects based on first test data. Each of the ability values is an ability value relating to a motor function of a body of a corresponding one of the first subjects. The first test data may describe a test result relating to the motor function of each of the first subjects. The test result may include determination values, for respective test items, for a corresponding one of the first subjects.

The alternative information processing system may comprise a calculation formula setter configured to set a calculation formula for a score corresponding to an ability value in a respective one of attribute classes included in a subject's attribute, based on a distribution of the ability values calculated by the ability value calculator in the respective one of the attribute classes.

The alternative information processing system may comprise a score calculator configured to calculate the score corresponding to an ability value of a second subject relating to the motor function based on the ability value of the second subject and on the calculation formula in one of the attribute classes corresponding to the second subject. The ability value may be calculated based on second test data. The second test data may include determination values, for the respective test items relating to the motor function of the second subject.

The alternative information processing system may comprise an outputter configured to output the score calculated by the score calculator. Setting the calculation formula in each attribute class based on the distribution in each attribute class makes it possible to adequately express with a score the level of the motor function taking into account the subject's attribute.

According to one aspect of the present disclosure, the calculation formula setter may set the calculation formula in each attribute class based on a standard value of the ability values of a subject group in a corresponding attribute class and on a standard deviation of the ability values of the subject group in the corresponding attribute class such that a specific range of the score is allocated to a range of the ability values that has a width proportional to the standard deviation centered at the standard value.

According to the method of allocating the score to the ability value while taking into account the standard deviation, it is possible to reduce variations in the ranges of scores among the attribute classes caused by variations in the standard deviations among the attribute classes.

According to one aspect of the present disclosure, the subject's attribute may be defined by an age of a subject. The calculation formula may be a calculation formula for calculating, from the ability value, a health age relating to the motor function as the score. The calculation formula may be configured to calculate a health age of the second subject such that the health age of the second subject whose ability value is the standard value corresponds to an actual age of that second subject.

According to one aspect of the present disclosure, the subject's attribute may be defined by a combination of the age and a sex.

According to one aspect of the present disclosure, the calculation formula setter may derive a regression model based on a regression analysis using, as samples, the ability values of the first subjects, the regression model including an explanatory variable corresponding to the subject's attribute and an objective variable corresponding to the ability value, the regression model defining the standard value of the ability values in each attribute class; and set the calculation formula so as to allocate, based on the standard value of the ability values and on a standard deviation corresponding to a variation from the standard value of the ability values of the first subjects, a range of the ability values that has a width proportional to the standard deviation centered at the standard value to the specific range of the score, in each attribute class.

According to one aspect of the present disclosure, the ability values may be values calculated in accordance with an item response model based on an item response theory, the item response model using the ability value of a subject and a difficulty level of a respective one of the test items and thereby representing an occurrence probability relating to each determination value that possibly takes for the respective one of the test items.

Taking into account the difficulty level of each test item allows an appropriate ability value to be calculated, synthesizing determination values in two or more test items, as the ability value relating to the motor function. Accordingly, it is possible to calculate an appropriate score corresponding to the motor function of the subject.

According to one aspect of the present disclosure, a computer program may be provided to cause a computer to function as at least a portion of the ability value calculator, the calculation formula setter, the score calculator, and the outputter in any one of the information processing systems described above. According to one aspect of the present disclosure, by causing the computer to perform the information processing method, the functions of the information processing system may be performed.

According to one aspect of the present disclosure, an information processing method implemented by a computer may be provided. The information processing method may comprise calculating ability values of two or more first subjects based on first test data. Each of the ability values is an ability value relating to a motor function of a body of a corresponding one of the first subjects. The first test data may describe a test result relating to the motor function of each of the first subjects. The test result may include determination values, for respective test items, for a corresponding one of the first subjects.

The information processing method may comprise setting a calculation formula for a score corresponding to an ability value based on a distribution of the ability values calculated relative to a subject's attribute. The information processing method may comprise calculating the score corresponding to an ability value of a second subject relating to the motor function based on the ability value of the second subject and on the calculation formula. The ability value of the second subject may be calculated based on second test data. The second test data may include determination values, for the respective test items relating to the motor function of the second subject.

The information processing method may comprise outputting the calculated score. The subject's attribute may be defined by an age of a subject. The calculation formula may be a calculation formula for calculating, from the ability value, a health age relating to the motor function as the score. Setting the calculation formula may comprise setting the calculation formula in accordance with a regression model between the age and the ability value, using a regression analysis using, as samples, the ability values of the first subjects.

According to one aspect of the present disclosure, an alternative information processing method implemented by a computer may be provided.

The alternative information processing method may comprise calculating ability values of first subjects based on first test data. Each of the ability values are an ability value relating to a motor function of a body of a corresponding one of the first subjects. The first test data may describe a test result relating to the motor function of each of the first subjects. The test result may include determination values, for respective test items, for a corresponding one of the first subjects.

The alternative information processing method may comprise setting a calculation formula for a score corresponding to an ability value in a respective one of attribute classes included in a subject's attribute, based on a distribution of the ability values calculated in the respective one of the attribute classes.

The alternative information processing method may comprise calculating the score corresponding to an ability value of a second subject relating to the motor function based on the ability value of the second subject and on the calculation formula in one of the attribute classes corresponding to the second subject. The ability value of the second subject may be calculated based on second test data. The second test data may include determination values, for the respective test items relating to the motor function of the second subject. The alternative information processing method may comprise outputting the score calculated.

According to the alternative information processing method, setting the calculation formula may comprise setting the calculation formula in each attribute class based on a standard value of the ability values of a subject group in a corresponding attribute class and on a standard deviation of the ability values of the subject group in the corresponding attribute class such that a specific range of the score is allocated to a range of the ability values that has a width proportional to the standard deviation centered at the standard value.

According to the alternative information processing method, the ability values may values calculated in accordance with an item response model based on an item response theory, the item response model using the ability value of a subject and a difficulty level of a respective one of the test items and thereby representing an occurrence probability relating to each determination value that possibly takes for the respective one of the test items.

According to a still another aspect of the present disclosure, a computer program may be provided to cause a computer to perform any one of the information processing methods described above. A non-transitory computer readable recording medium may be provided in which a computer program including instructions to cause a computer to perform the information processing method.

### BRIEF DESCRIPTION OF THE DRAWINGS

FIG. 1 is a block diagram of a configuration of an information processing system.

FIG. 2 is a diagram of a configuration of subject data.

FIG. 3 is a flowchart (Part 1) of analytical processing executed by a processor in a first embodiment.

FIG. 4 is a flowchart (Part 2) of the analytical processing in the first embodiment.

FIG. 5A is a graph showing an example distribution of ability values relating to a group of male subjects.

FIG. 5B is a graph showing an example distribution of corresponding individual characteristic values.

FIG. 6A is a graph showing an example distribution of ability values relating to a group of female subjects.

FIG. 6B is a graph showing an example distribution of corresponding individual characteristic values.

FIG. 7 is a graph explaining a relation between the distribution of individual characteristic values and a range of health age.

FIG. 8 is a block diagram of a configuration of a system of providing information on health age from a web server.

FIG. 9 is a flowchart of service provision processing executed by a processor of the web server.

FIG. 10 is a diagram explaining an example of a service provision screen displaying on a communication terminal.

FIG. 11 is a flowchart of portion of analytical processing of a second embodiment.

FIG. 12 is a flowchart of analytical processing of a third embodiment.

FIG. 13 is a graph explaining a relation between an actual age and health age.

### EXPLANATION OF REFERENCE NUMERALS

1 . . . information processing system, 11 . . . processor, 13 . . . memory, 15 . . . storage, 17 . . . user interface, 19 . . . communication interface, 50 . . . web server, 51 . . . processor, 53 . . . memory, 55 . . . storage, 60 . . . communication terminal, 70 . . . service provision screen, 71 . . . input field, 75 . . . display field.

### MODE FOR CARRYING OUT THE INVENTION

Hereinafter, an example embodiment of the present disclosure will be explained in detail with reference to the drawings.

### First Embodiment

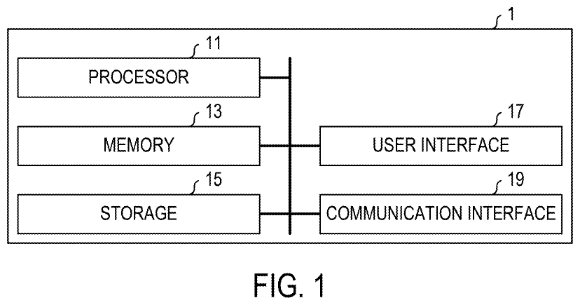

An information processing system 1 of the present embodiment shown in FIG. 1 is configured to calculate health age relating to lower limb motor function. A formula for calculating the health age is derived from test results relating to lower limb motor functions of two or more subjects using a statistical approach.

The information processing system 1 comprises a computer system with a dedicated computer program installed. The information processing system 1 comprises a processor 11, a memory 13, a storage 15, a user interface 17, and a communication interface 19.

The processor 11 executes a processing in accordance with the computer program. The memory 13 comprises a RAM and is used as workspace during the processing executed by the processor 11. The storage 15 comprises a hard disk drive and/or a solid-state drive, and stores the computer program and a variety of data.

The user interface 17 comprises an input device such as a keyboard and mouse, and a display device such as a liquid crystal display. The user interface 17 is configured to be capable of receiving operation from a user and displaying a variety of information to the user. The communication interface 19 is configured to be communicable with an external device.

In order to derive the calculation formula and further to calculate the health age, the information processing system 1 acquires, from an external device, subject data including a test result of each subject in a subject group, and stores, in the storage 15, the acquired subject data of each subject.

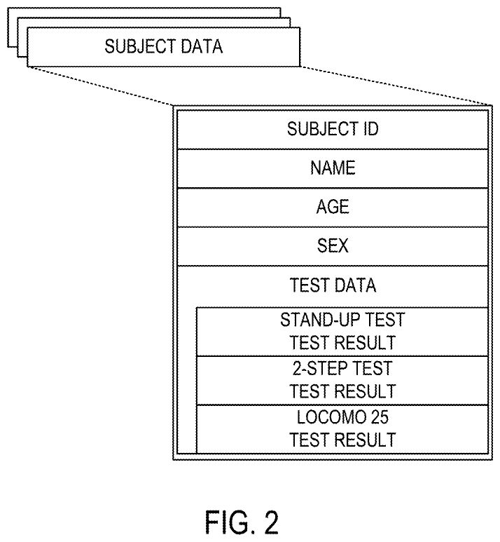

According to an example shown in FIG. 2, the subject data comprises not only data indicating an identification code, a name, an age, and a sex of a corresponding subject, but also test data relating to the lower limb motor function of the corresponding subject. Hereinafter, the identification code of the subject is referred to as a subject ID. The test data describes a determination value of each of test items obtained from a test relating to the lower limb motor function that the corresponding subject has been taken.

The test relating to the lower limb motor function is performed, for example, using three tests: “stand-up test”, “2-step test”, and “Locomo 25” recommended by The Japanese Orthopedic Association. The test data describes, for example, a determination value of each test item relating to the three tests.

The “stand-up test” is a test for determining lower limb muscle strength. In this test, a variety of stools including seat surfaces with different heights are provided. Specifically, the stools including seat surfaces with the heights of 10 cm, 20 cm, 30 cm, and 40 cm are provided. The lower the height of the seat surface of the stool is, the greater the lower limb muscle strength required to stand up from a seated position on this stool. The lower limb muscle strength required to stand up on one leg is greater than that on both legs.

In the “stand-up test”, the subject stands up on one leg from a seated position while switching from a stool with a higher seat surface to a stool with a lower seat surface. If the subject cannot stand up on one leg, then a test is performed such that the subject stands up on both legs from the seated position of the seat surface having the same height. In this manner, a magnitude of the lower limb muscle strength is determined based on nine-level assessments. The test data can describe a determination value of the lower limb muscle strength as a test result relating to the “stand-up test”. The determination value is an integer value ranging from a value of 0 to a value of 8.

The “2-step test” is a test for determining walking ability. In this test, a stride length of two steps when the subject walks under a specific condition is measured. A two-step value is calculated by dividing the stride length of the two steps by the height of the subject. The test data can describe the two-step value as the test result of the “2-step test”.

The “Locomo 25” is a test in questionnaire form in which the subject answers 25 questions relating to physical pain and difficulties that have happened in daily lives over a one month. Each of the 25 questions corresponds to one of the test items relating to the lower limb motor function.

The 25 questions include a question “Do you have pain anywhere in your lower limbs?”, and the answer options to this question: “No pain”, “Slight pain”, “Moderate pain”, “Considerable pain,” and “Severe pain” are provided. The 25 questions further include a question “How difficult is it for you to walk inside your house?”, and the answer options to this question: “Not difficult,” “Slightly difficult,” “Moderately difficult,” “Considerably difficult,” and “Extremely difficult” are provided.

According to the “Locomo 25”, a five-level answer to each of the 25 questions can be obtained from the subject. The test data describes the answer to each of the 25 questions from the subject in a numerical value as the test result relating to the “Locomo 25”. The answer to each question corresponds to a determination value of each test item. The determination value is an integer value ranging from a value of 0 to a value of 4.

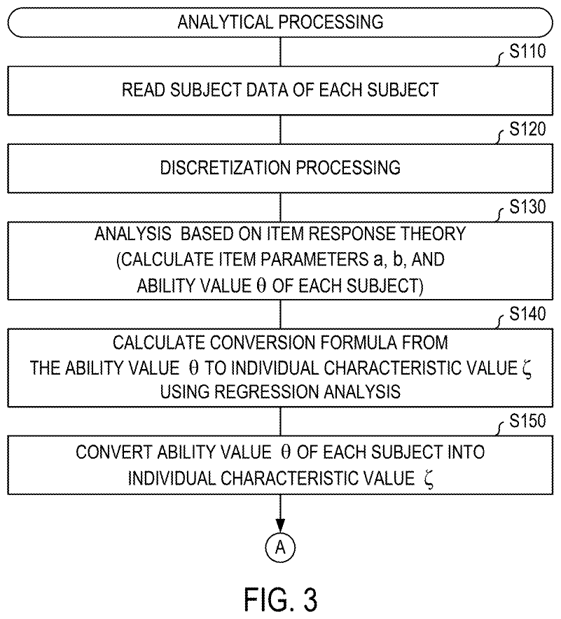

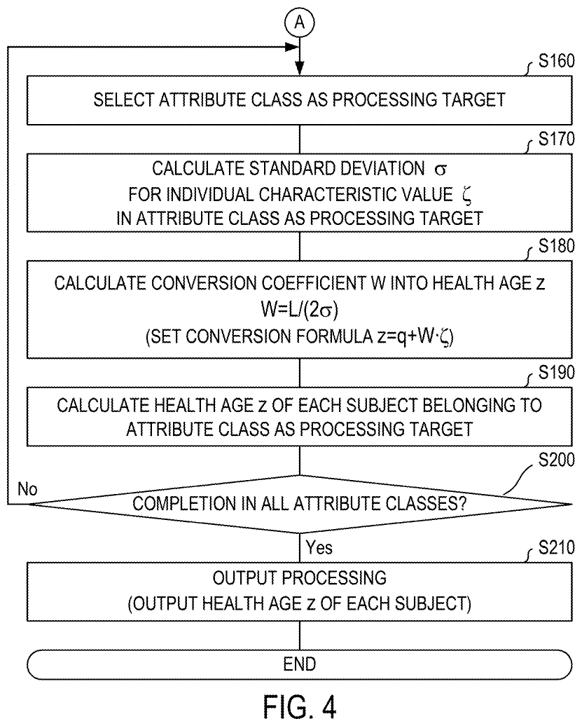

When an execution instruction for analytical processing is input, the processor 11 executes the analytical processing as shown in FIG. 3 and FIG. 4, in order to derive a formula for calculating the health age relating to the lower limb motor function. The execution instruction is input, for example, from a user of the information processing system 1 through the user interface 17.

When the analytical processing shown in FIG. 3 and FIG. 4 is started, the processor 11 reads from the storage 15 the subject data of each subject in the subject group specified by the user (S110), and executes discretization processing (S120).

In the discretization processing, the processor 11 converts the two-step value of each subject into a discrete value in accordance with a specific rule. The processor 11 discretizes the two-step value into a multi-level value, for example, in accordance with a conversion table stored in the storage 15.

A range of possible two-step values is equally or unequally classed into classes by a designer in advance. Each class corresponds to one level. The conversion table defines a relation between each class of the two-step values and a corresponding level. Accordingly, the conversion table defines the multiple levels for the two-step values. The discretized two-step value takes on integer values from values of 0 to M. M may be an integer value equal to or more than 1, in particular, an integer value corresponding to a maximum level.

Subsequently, in S130, the processor 11 analyzes the test results of the subject group based on item response theory (S130). Specifically, the test results of the subject group are analyzed using a partial credit model (PCM: Partial Credit Model), which is one of multi-level item response models.

In S130, a probability model in accordance with the following Formula (1) is used to calculate values for item parameters a and b that are likelihood for the test results of the subject group. In detail, the item parameters a and b are given where a set a={aᵢ|i=1, . . . , N} and a set b={bᵢ|i=1, . . . , N}.

$$[ Mathematical Formula 1 ]$$

$$P ( {Y}_{ij} = y \vert {θ}_{j} , {a}_{i} , {b}_{i} ) = \frac{exp {\sum }_{s = 1}^{y} {a}_{i} ( {θ}_{j} - {b}_{is} )}{1 + {\sum }_{k = 1}^{{m}_{i}} exp {\sum }_{s = 1}^{k} {a}_{i} ( {θ}_{j} - {b}_{is} )} \qquad ( 1 )$$

Index i corresponds to an item number. The item number corresponds to an identification number of the test item. Hereinafter, a test item with an item number i is referred to as i^{th} test item. N corresponds to the number of items.

Index j corresponds to the identification number of the subject, in other words, the subject ID. Hereinafter, a subject with an identification number j is referred to as a j^{th} subject. Yᵢⱼ corresponds to a response value of the i^{th} test item obtained from the j^{th} subject. The response value of the i^{th} test item corresponds to a determination value of the i^{th} test item. Yᵢⱼ=y means that the response value Yᵢⱼ of the i^{th} test item obtained from the j^{th} subject is a value of y.

For example, when the test item is the “2-step test”, the response value is the discretized two-step value described above. When the test item relates to the “stand-up test” or “Locomo 25”, the response value is the same as the determination value described above.

θⱼ is an ability value relating to a lower limb motor function of the j^{th} subject. aᵢ corresponds to an identification ability for the i^{th} test item. bᵢ corresponds to a difficulty level for the i^{th} test item, in detail, a set bᵢ={bᵢₛ|s=1, . . . , mᵢ} of category class values bᵢₛ.

mᵢ corresponds to the number of categories of the i^{th} test item. The number of categories is 1 less than a number of the response values that the i^{th} test item may take. Specifically, the number of categories of the test item in the “stand-up test” is 8, the number of categories of the test item in the “2-step test” is 41, and the number of categories of each test item in the “Locomo 25” is 4. The aforementioned probability model in the Formula (1) where the response value Yᵢⱼ=y=0 can be expressed by the following Formula (2).

$$[ Mathematical Formula 2 ]$$

$$P ( {Y}_{ij} = 0 \vert {θ}_{j} , {a}_{i} , {b}_{i} ) = \frac{1}{1 + {\sum }_{k = 1}^{{m}_{i}} exp {\sum }_{s = 1}^{k} {a}_{i} ( {θ}_{j} - {b}_{is} )} \qquad ( 2 )$$

The probability models expressed by the Formula (1) and the Formula (2) are item response models representing an occurrence probability relating to each determination value on which the i^{th} test item may take, using the ability value θⱼ of a subject, and the identification ability aᵢ and the difficulty level bᵢ of the i^{th} test item (in detail, the difficulty level bᵢₛ for each determination value).

In S130, based on a response value set {Yᵢⱼ} specified from the test data of the subject group, the values of the item parameters a and b that maximize a value of a likelihood function corresponding to a product of conditional probabilities P(Yᵢⱼ=y|θⱼ,aᵢ,bᵢ) are calculated. Furthermore, for each subject, based on the calculated values of the item parameters a and b and the response value Yᵢⱼ of each test item of the subject, the ability value θⱼ of the subject is calculated.

Thereafter, the processor 11 executes a regression analysis using, as samples, an ability value set {θⱼ} of the subject group as well as information of the sex and age of each subject, thereby calculating the following regression equation including explanatory variables x1, x2, and x3 relating to subject's attributes and an objective variable relating to the ability value θ (S140).

$$θ = f ( x 1 , x 2 , x 3 ) = γ 0 + γ 1 \cdot x 1 + γ2 \cdot x 2 + γ3 \cdot x 3$$

According to the present embodiment, the subject's attribute is defined by a combination of age and sex. The subject's attribute includes two or more attribute classes. Each attribute class corresponds to one of combinations of possible values, as a combination of age and sex. The subject's attribute includes, for males, an attribute class corresponding to each age, and for females, an attribute class corresponding to each age.

The explanatory variable x1 corresponds to the sex of the subject, and the explanatory variable x2 corresponds to the age of the subject. The explanatory variable x3 corresponds to an attribute class of the subject, specifically a class of a combination of sex and age thereof. γ0, γ1, γ2, γ3 correspond to regression coefficients.

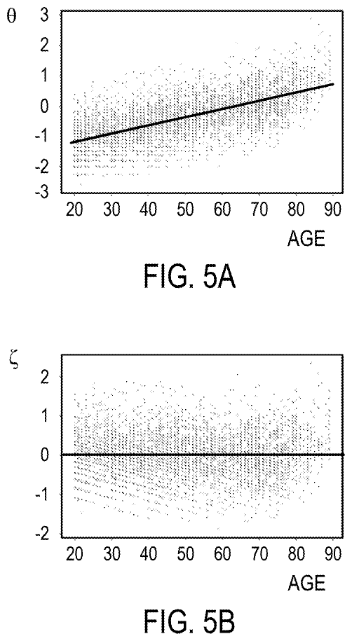

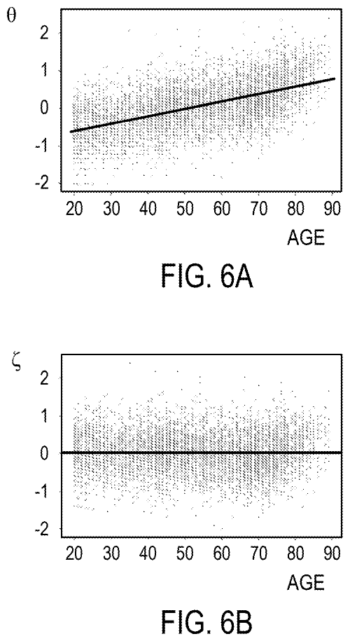

FIG. 5A shows an example of a distribution of the ability values θ relating to a group of male subjects on a graph with a horizontal axis representing age and a vertical axis representing the ability value θ. FIG. 6A shows an example of a distribution of the ability values θ relating to a group of female subjects on a graph with a horizontal axis representing age and a vertical axis representing the ability values θ. Straight lines shown in FIG. 5A and FIG. 6A represent regression lines in dimensions of the age versus the ability value.

The regression analysis is performed by searching for the regression coefficients γ0, γ1, γ2, γ3 that minimize a sum of squared error Σ|θⱼ−f(x1, x2, x3)|². The ability value θ calculated using regression equation θ=f(x1, x2, x3) including the searched regression coefficients γ0, γ1, γ2, γ3 corresponds to a standard ability value of the subject group that is a group of subjects each having the sex and age represented by x1, x2, x3. Hereinafter, the standard ability value θ calculated by the regression equation is expressed as, in particular, a standard ability value θr.

The item of sex (γ1·x1) included in the regression equation represents a sex component included in the standard ability value θr, the item of age (γ2·x2) included in the regression equation represents an age component included in the standard ability value θr, and the item of sex and age (γ3·x3) represent a sex and age component included in the standard ability value θr. γ0 corresponds to an intercept, and represents a basic component that does not rely on the sex and age in the standard ability value θr.

A difference (θj−θr) of the ability value θⱼ of each subject with respect to the standard ability value θr corresponds to the ability value of the subject when the standard value of the ability value is set to zero for the sex and age of the subject. Hereinafter, the ability value (θⱼ−θr) is expressed as an individual characteristic value ζⱼ.

The individual characteristic value ζⱼ of each subject can be calculated by the formula ζⱼ=θⱼ−f(x1, x2, x3), based on the ability value θⱼ of the corresponding subject and on the sex and age of the corresponding subject.

In S140, as a conversion formula from the ability value θ to the individual characteristic value ζ, ζ=g(θ, x1, x2, x3)=ζ−f(x1, x2, x3) is set. Subsequently, in S150, the processor 11 converts the ability value θⱼ of each subject to the individual characteristic value ζⱼ=g(θ=θⱼ, x1, x2, x3), based on the sex and age of the corresponding subject.

FIG. 5B shows an example of a distribution of individual characteristic values ζ of the group of male subjects on a graph with a horizontal axis representing age and a vertical axis representing the individual characteristic value ζ. FIG. 6B shows an example of a distribution of individual characteristic values ζ of the group of female subjects on a graph with a horizontal axis representing age and a vertical axis representing the individual characteristic value ζ. Straight lines shown in FIG. 5B and FIG. 6B are straight lines where the individual characteristic value ζ=0.

Thereafter, the processor 11 executes, for each attribute class, processing of converting the individual characteristic value ζⱼ of the corresponding subject to a health age zⱼ (S160-200). In S160, the processor 11 selects an attribute class as a processing target. Specifically, the processor 11 selects sex and age as the processing target. Thereafter, the processor 11 identifies a distribution of individual characteristic values {ζⱼ} of the subject group in the selected attribute class as the processing target. In detail, the processor 11 calculates a standard deviation σ for the individual characteristic values {ζⱼ} of the subject group in the selected attribute class as the processing target (S170).

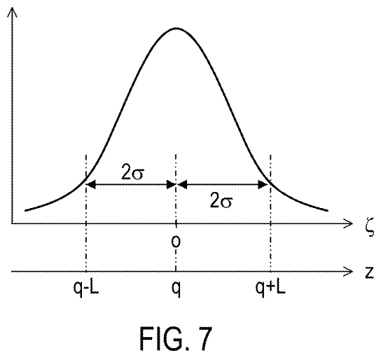

Subsequently, in S180, the processor 11 sets a conversion formula z=h(q, ζ)=q+W·ζ from the individual characteristic value ζ into the health age z. q is an actual age, and W is a conversion coefficient. As shown in FIG. 7, the processor 11 sets a conversion coefficient in accordance with a formula W=L/(2σ) using the standard deviation σ calculated in S170 such that a range of variation of the health age z falls within a range of +L from the actual age q at 95% confidence interval. Specifically, the processor 11 sets the conversion formula for converting the individual characteristic value ζ into the health age z, to z=h(q, ζ)=q+{L/(2σ)}·ζ, with respect to the attribute class as the processing target.

Subsequently, in S190, the processor 11 uses the conversion formula z=h (q, ζ) set in S180 to convert the individual characteristic value ζⱼ of each subject in the attribute class as the processing target into the health age zⱼ=qⱼ+W·ζⱼ.

As described, the health age zⱼ of j^{th} subject is a value where an age adjustment+W·ζⱼ in accordance with the individual characteristic value ζⱼ is added to an actual age qⱼ of the j^{th} subject. If the health age z calculated in accordance with the conversion formula z=h (q, ζ) exceeds the range of ±L from the actual age q, the processor 11 may modify the health age z to z=q+L or z=q−L. Specifically, the health age z may be calculated within the range of ±L from the actual age q.

Until the conversion formula z=h(q, ζ) for each attribute class is set with respect to all attribute classes, the processor 11 makes a negative determination in S200 and repeatedly executes the processing in S160 through S190. This allows the processor 11 to set the conversion formula z=h(q, ζ) for each attribute class and calculate the health age zⱼ of each subject using the corresponding conversion formula z=h(q, ζ) in the attribute class.

When completing the calculation of the conversion formula z=h(q, ζ) for each attribute class and the calculation of the health age z with respect to all attribute classes, the processor 11 makes an affirmative determination in S200 and executes output processing in S210. In the output processing, the processor 11 can prepare a report for each subject that explains a lower limb motor ability of the corresponding subject along with the health age z, and output the report to each subject.

For example, the processor 11 can email the report of each subject to the subject's email address that has been registered in advance. Alternatively, the processor 11 can execute processing of outputting such a report through a printer as an external device, in order to provide each subject with the report via paper medium.

The report may be registered on a web server provided for a review of the report. In accordance with a request from a communication terminal of each subject, the web server can send the corresponding report as a web page to the communication terminal.

The probability model including the item parameters a and b that are derived in the analytical processing of the information processing system 1, the conversion formula ζ=g(θ, x1, x2, x3) to the individual characteristic value ζ, and the conversion formula z=h(q, ζ) for each attribute class may be used to calculate the health age z of a subject other than the subject group used for deriving these conversion formulas.

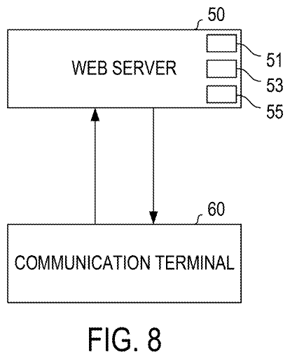

For example, a web server 50 that provides a service for calculating the health age may be established as shown in FIG. 8. As shown in FIG. 8, the web server 50 may comprise a processor 51, a memory 53, and a storage 55. The storage 55 can store the probability model derived in the analytical processing of the information processing system 1, the conversion formula ζ=g(θ, x1, x2, x3), and the conversion formula z=h(q, ζ).

As used herein, the web server 50 is a different computer system from the information processing system 1, but the information processing system 1 may function as the web server 50.

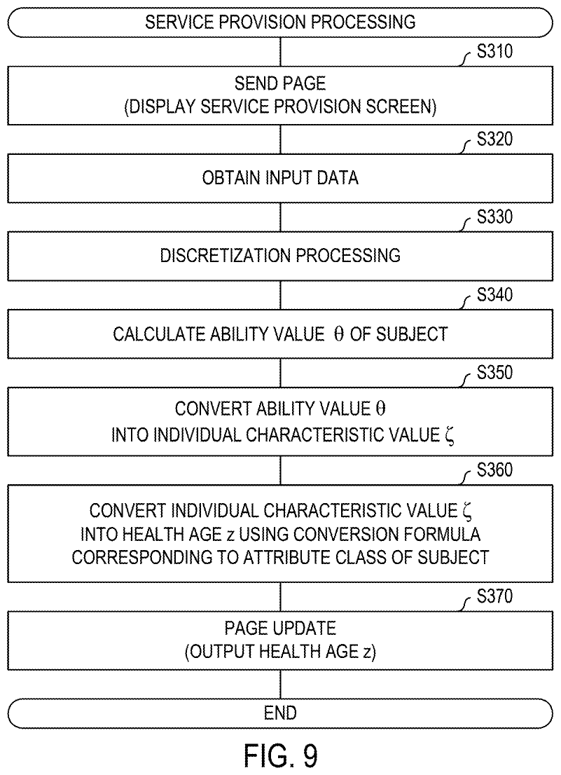

The processor 51 of the web server 50 can execute service provision processing shown in FIG. 9 if a web page providing a calculation service is accessed from a communication terminal 60 of a user.

When starting the service provision processing, the processor 51 sends, to the communication terminal 60 as an access source, a web page for displaying a service provision screen 70 on the communication terminal 60 (S310). The service provision screen 70 includes an input field 71 for input of the test result relating to the lower limb motor function along with the sex and age of the subject.

The communication terminal 60 displays the service provision screen 70 in response to receipt of the web page, and receives an input operation relative to the input field 71. This allows the user of the communication terminal 60 to input the user's own test result through the service provision screen 70 displayed on the communication terminal 60.

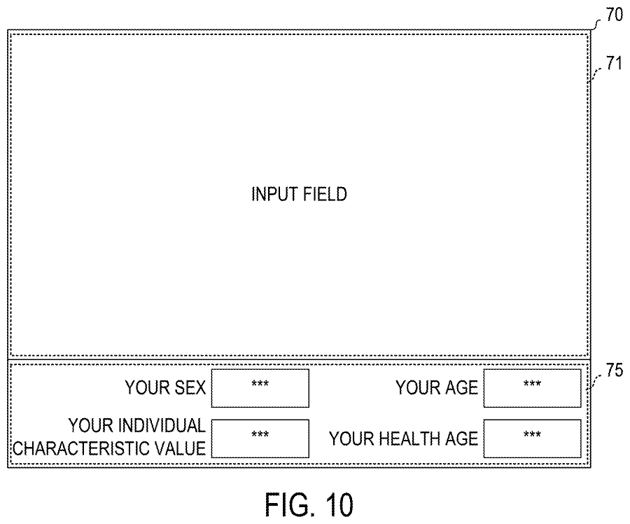

The service provision screen 70, which is shown as an example in FIG. 10, comprises a display field 75 in addition to the input field 71. The input field 71 is configured to allow input of the test result relating to the lower limb motor function of the subject along with the sex and age of the subject through the communication terminal 60.

Specifically, the input field 71 is configured to allow, as the test result, input of the determination values of the two or more test items relating to the “stand-up test”, “2-step test”, and “Locomo25”. The user can perform the tests for the lower limb motor function on his/her own through the “stand-up test”, “2-step test”, and “Locomo25”, and input the results in the input field 71.

The display field 75 is an area for displaying information provided by the web server 50 based on the test results that have been input to the input field 71. The display field 75 is configured to display the sex, age, individual characteristic value ζ, and health age z of the subject.

After sending the web page to the communication terminal 60 in S310, the processor 51 receives input data describing the sex, age, and test result of the subject sent from the communication terminal 60, by completion of the input operation through the service provision screen 70 (S320).

Subsequently, in S330, the processor 51 discretizes the two-step value included in the input data into a multi-level value. Subsequently, in S340, the processor 51 calculates the ability value θ of the user, based on the input data and the probability model of the following formula (3) that includes the item parameters a and b.

$$[ Mathematical Formula 3 ]$$

$$P ( {Y}_{i} = y \vert θ , {a}_{i} , {b}_{i} ) = \frac{exp {\sum }_{s = 1}^{y} {α}_{i} ( θ \to {b}_{is} )}{1 + {\sum }_{k = 1}^{{m}_{i}} exp {\sum }_{s = 1}^{k} {a}_{i} ( θ - {b}_{i s} )} \qquad ( 3 )$$

The processor 11 can calculate, as the ability value θ, a value θ that maximizes the likelihood function corresponding to a product of the conditional probabilities P(Yᵢ=y|θ, aᵢ, bᵢ) based on the set of determination values relating to the two or more test items included in the input data (S340).

Thereafter, the processor 51 inputs the calculated ability value θ into the conversion formula ζ=g (θ, x1, x2, x3) and converts the ability value θ into the individual characteristic values ζ. To the conversion formula ζ=g (θ, x1, x2, x3), the values corresponding to the sex and age of the user specified from the input data are input as x1, x2, x3 (S350).

Furthermore, the processor 51 inputs the calculated individual characteristic value ζ to the conversion formula z=h(q, ζ)=q+{L/(2σ)}·ζ corresponding to the attribute class of the user, specifically, the sex and age of the user, among the conversion formulas for respective attribute classes, and converts the individual characteristic value ζ to the health age z. This allows the processor 51 to calculate the health age z of the user (S360).

Thereafter, the processor 51 sends, to the communication terminal 60, an updated web page displaying the sex and age of the user and the calculated individual characteristic value ζ and health age z in the display field 75 (S370). As a result, information including the individual characteristic value ζ relating to the lower limb motor function of the user and the health age z is provided from the web server 50 to the communication terminal 60 of the user. The communication terminal 60 that has received the web page displays an updated service provision screen 70, thereby providing the user with the information on the individual characteristic value ζ relating to the lower limb motor function and the health age z.

As above, the configurations of the information processing system 1 and the web server 50 in the first embodiment are explained. According to the present embodiment, the processor 11 of the information processing system 1 functions as an ability value calculator, and calculates the ability value θ relating to the motor function of each subject based on a subject data set of the subject group (S130).

The processor 11 further functions as a calculation formula setter, and sets the conversion formula ζ=g(θ, x1, x2, x3) and the conversion formula z=h(q, ζ)=q+{L/(2σ)}·ζ based on the distribution of the ability values θ for the subject's attributes, as a calculation formula for the health age z that is a score corresponding to the ability value θ (S140, S180).

The processor 11 further functions as a score calculator, and calculates, based on the ability value θ of each subject in the subject group and on the aforementioned calculation formulas, the health age z corresponding to the ability value θ of each subject (S150, S190). The processor 11 further functions as an outputter and outputs the calculated health age z (S210).

The processor 51 of the web server 50 also functions as a score calculator and as an outputter, and calculates and outputs, based on data indicating the determination value of each test item relating to a lower limb motor function of a subject other than the subject group, the health age z of the subject (S360, S370).

According to the present embodiment, since the health age z is calculated taking into account the distribution of the ability values θ of the subjects for each attribute class, the subject can easily determine, based on the health age z that has been informed, whether the level of his/her own motor function taking into account the sex and age is high or low. Therefore, according to the present embodiment, it is possible to establish a system useful for health management of subjects, and can provide corresponding services.

According to the present embodiment, in particular, based on the regression analysis for the ability value set {θⱼ}, the aforementioned regression equation is calculated as the regression model between the sex and age, and the ability value θ. The ability value θ in accordance with the regression equation is treated as the standard ability value θr, a difference of the ability value θ of each individual from the standard ability value θr is calculated as the individual characteristic value ζ. The health age z is calculated based on the individual characteristic value ζ.

The calculation of the individual characteristic value ζ and the calculation of the health age z using the regression analysis make it possible to appropriately set the standard value for the ability value θ for each attribute class from a statistical perspective, and calculate an appropriate health age z taking into account the standard value.

According to the present embodiment, for each attribute class, the calculation formula for the health age z is prepared taking into account the standard deviation σ representing the distribution characteristic of the individual characteristic values ζ. To the range −2σ≤ζ≤+2σ of the individual characteristic value ζ that has a width proportional to the standard deviation σ centered at the individual characteristic value ζ=0 corresponding to the standard ability value θr, the health age z in a range of q−L≤z≤q+L that has a specific width centered at the actual age q is allocated. To a subject whose individual characteristic value ζ is zero, that is, whose ability value θ is the standard ability value θr, the health age z=q equal to the actual age q is allocated.

According to the method of allocating, for each attribute class, the health ages z to the individual characteristic values ζ taking into account the standard deviations σ, it is possible to reduce variations in the range of the health ages z caused by variations in the standard deviations σ among the attribute classes. Accordingly, it is possible to inform the subjects about the health ages z that are comparable among different attribute classes. This can provide each subject with the health age z, allowing subjects to easily understand changes in their lower limb motor functions over years. Furthermore, it is possible to provide the health age z that are comparable among family members and friends.

In addition, according to the present embodiment, the ability value θ relating to the lower limb motor function is calculated taking into account the difficulty level of each test item and the difficulty level of each determination value using the item response model. Therefore, it is possible to calculate an appropriate ability value θ obtained by synthesizing determination values of the two or more test items, and calculate an appropriate health age z based on the appropriate ability value θ.

### Second Embodiment

Subsequently, a configuration of the information processing system 1 in a second embodiment will be explained. The information processing system 1 in the second embodiment is configured similarly to the information processing system 1 in the first embodiment, except that analytical processing executed by the processor 11 in the second embodiment partly differs from that in the first embodiment. Hereinafter, in the following explanation of the information processing system 1 in the second embodiment, a configuration different from the first embodiment is selectively explained and the explanation of the same configuration will be omitted as appropriate.

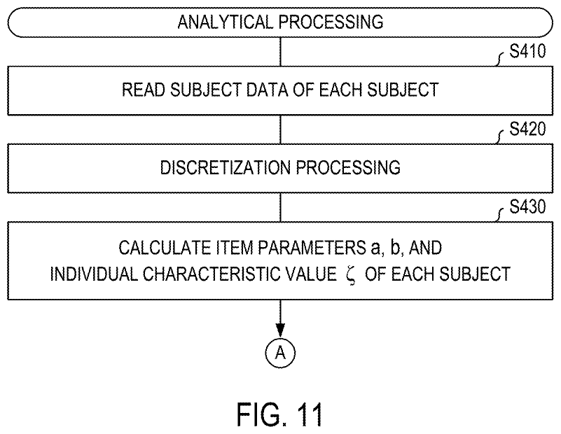

The processor 11 executes analytical processing in FIG. 11 instead of the analytical processing shown in FIG. 3. When this analytical processing is started, the processor 11 reads from the storage 15 the subject data of each subject in the subject group specified by the user (S410), and executes discretization processing (S420), similarly to the processing in S110 and S120 in the first embodiment.

Subsequently, in S430, the processor 11 calculates the item parameters a and b and the coefficients γ0, γ1, γ2, γ3, and the individual characteristic value ζⱼ of each subject, in accordance with a probability model in which the ability value θⱼ of the probability model according to the Formula (1) in the first embodiment is replaced with θⱼ=γ0+γ1·x1ⱼ+γ2·x2ⱼ+γ3·x3ⱼ+ζⱼ. x1ⱼ is a sex of the j^{th} subject, x2ⱼ is an age of the j^{th} subject, and x3ⱼ is a category of sex and age of the j^{th} subject. Thereafter, the processor 11 executes processing in S160 to S210, similarly to the first embodiment.

Also, by the method of calculating the individual characteristic values ζⱼ of each subject using the above-described method and converting the individual characteristic values ζⱼ into the health age zⱼ by the above-described method, similar effects to the effects in the first embodiment can be obtained. In accordance with the configuration of the information processing system 1 in the present embodiment, the processor 51 of the web server 50 can execute the following processing in the service provision processing of S340 and S350.

Specifically, in S340 and S350, the processor 51 can calculate the individual characteristic value ζ, taking into account the sex and age of the subject, based on the probability model used in the processing in S430 and on the calculated item parameters a and b and coefficients γ0, γ1, γ2, γ3.

### Third Embodiment

Subsequently, a configuration of the information processing system 1 in a third embodiment will be explained. The information processing system 1 in the third embodiment is configured similarly to the information processing system 1 in the first embodiment, except that analytical processing executed by the processor 11 in the third embodiment partly differs from that in the first embodiment. Hereinafter, in the following explanation of the information processing system 1 in the third embodiment, a configuration different from the first embodiment is selectively explained and the explanation of the same configuration will be omitted as appropriate.

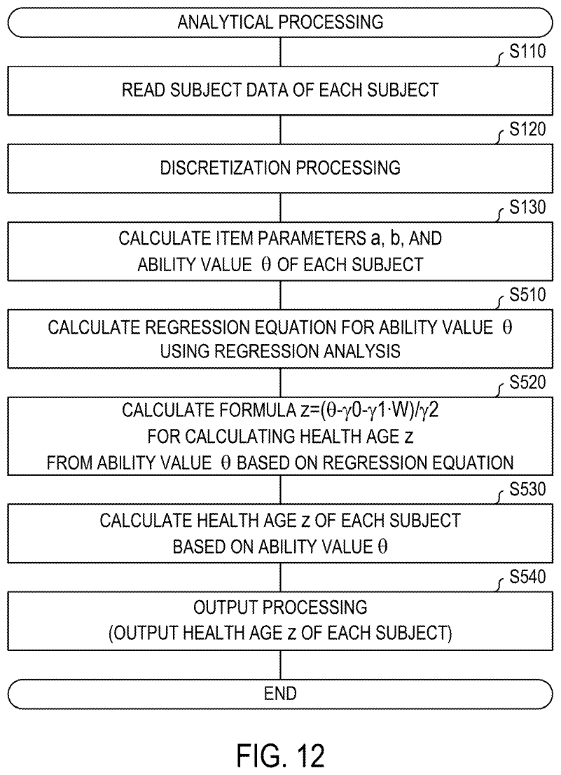

According to the third embodiment, the processor 11 executes analytical processing shown in FIG. 12, instead of the analytical processing shown in FIG. 3 and FIG. 4. When this analytical processing is started, the processor 11 executes the processing in S110-S130, similarly to that of the first embodiment.

Thereafter, the processor 11 derives, by a regression analysis, a calculation formula for calculating the health age z corresponding to the ability value θ.

Specifically, similarly to the processing in S140, the processor 11 executes the regression analysis using the ability value set {θⱼ} of a subject group along with information of the sex and age of each subject, thereby calculating the following regression equation where subject's attributes are explanatory variables x1, x2, and the ability value θ is an objective variable (S510).

$$θ = γ 0 + γ1 \cdot x 1 + γ2 \cdot x 2$$

The meaning of the regression coefficients γ0, γ1, γ2 and the explanatory variables x1, x2 is similar to those in the first embodiment. Herein, for a simpler description, an example in which the regression equation does not include the item of sex and age (γ3·x3) will be explained. However, the regression equation may include the item of sex and age (γ3·x3).

By transforming the regression equation, the processor 11 calculates a calculation formula z=(θ−γ0−γ1·W)/γ2 for calculating the health age z from the ability value θ for each sex (S520). A variable W represents sex.

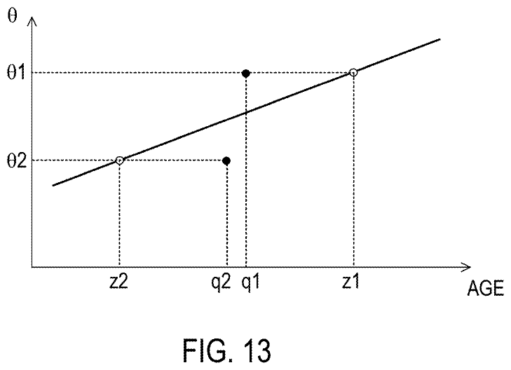

The processor 11 further substitutes the ability value θⱼ of each subject into the calculation formula in accordance with the sex of the subject, thereby calculating the health age zⱼ=(θⱼ−γ0−γ1·W)/γ2 of each subject (S530). A relation between the health age z calculated here and the actual age q is shown in FIG. 13.

If the actual age q=q1 and the ability value θ=θ1, the health age z is calculated as an age z1 where the standard ability value θr that is an ability value on a regression line is θ1. Similarly, if the actual age q=q2 and the ability value θ=θ2, the health age z is calculated as an age z2 where the standard ability value θr is θ2.

Thereafter, the processor 11 executes output processing (S540). In the output processing, the processor 11 can prepare a report for each subject that explains a lower limb motor ability of a corresponding subject along with the health age z, and output the report to each subject.

According to the third embodiment, it is possible to easily calculate the health age z without obtaining the distribution relating to sex and age in each attribute class, specifically, the standard deviation σ. Also, in the present embodiment, the web server 50 may execute the service provision processing in accordance with the configuration of the information processing system 1

That is, the processor 51 of the web server 50 can execute processing of calculating the health age z from the user's ability value θ in accordance with the calculation formula z=(θ−γ0−γ1·W)/γ2, in place of the processing in S350 and S360.

The example embodiments of the present disclosure have been described above, but it should be noted that the present disclosure should not be limited to the aforementioned embodiments and may be embodied in various modes.

For example, the item parameters a and b may be updated periodically based on subject data that has been stored by that time. The item parameters a and b may be updated every time when new subject data or test data are registered. The item parameter a corresponding to the identification ability does not have to be used. In this case, in the probability model of the Formulas (1) to (3), aᵢ (i=1, . . . , N) may be set to a fixed value of “1”.

The regression analysis in S140 is not limited to a linear regression. For example, a non-linear regression may be employed as the regression analysis in S140, and the function f(x1, x2, x3) and the conversion formula ζ=g(θ, x1, x2, x3) may each include a non-linear regression model. This idea can be applied to the replacement of the ability value θⱼ in S430 and the calculation of the regression equation in S510.

The technique of the present disclosure is not limited to the lower limb motor function but may be used to calculate a health age relating to other physical motor functions. For example, the technique of the present disclosure may be used to calculate a health age relating to an upper-body motor function.

In addition, one or more functions of one element in the aforementioned embodiments may be distributed to two or more elements. Functions of two or more elements in the aforementioned embodiments may be integrated into one element. A part of the configurations of the aforementioned embodiments may be omitted. At least a part of one configuration of the aforementioned embodiments may be added to or replaced with another configuration of the aforementioned embodiments. All aspects included in the technical ideas identified by the languages recited in the claims are embodiments of the present disclosure.

1. An information processing system, comprising:
an ability value calculator configured to calculate ability values of first subjects based on first test data, each of the ability values being an ability value relating to a motor function of a body of a corresponding one of the first subjects, the first test data describing a test result relating to the motor function of each of the first subjects, the test result including determination values, for respective test items, for a corresponding one of the first subjects;
a calculation formula setter configured to set a calculation formula for a score corresponding to an ability value based on a distribution of the ability values calculated by the ability value calculator relative to a subject's attribute;
a score calculator configured to calculate the score corresponding to an ability value of a second subject relating to the motor function based on the ability value of the second subject and on the calculation formula, the ability value being calculated based on second test data, the second test data including determination values, for the respective test items relating to the motor function of the second subject; and
an outputter configured to output the score calculated by the score calculator.

2. The information processing system according to claim 1,
wherein the subject's attribute is defined by an age of a subject, and
wherein the calculation formula is a calculation formula for calculating, from the ability value, a health age relating to the motor function as the score.

3. The information processing system according to claim 2,
wherein the calculation formula setter is configured to set the calculation formula in accordance with a regression model between the age and the ability value, based on a regression analysis using, as samples, the ability values of the first subjects.

4. The information processing system according to claim 1,
wherein the subject's attribute includes attribute classes,
wherein the calculation formula setter is configured to set a calculation formula for a score corresponding to an ability value in a respective one of the attribute classes, based on a distribution of the ability values calculated by the ability value calculator in the respective one of the attribute classes, and
wherein the score calculator is configured to calculate the score corresponding to the ability value of the second subject relating to the motor function based on the ability value of the second subject and on the calculation formula in one of the attribute classes corresponding to the second subject.

5. The information processing system according to claim 4, wherein the calculation formula setter is configured to set the calculation formula in each attribute class based on a standard value of the ability values of a subject group in a corresponding attribute class and on a standard deviation of the ability values of the subject group in the corresponding attribute class such that a specific range of the score is allocated to a range of the ability values that has a width proportional to the standard deviation centered at the standard value.

6. The information processing system according to claim 4,
wherein the subject's attribute is defined by an age of a subject, and
wherein the calculation formula is a calculation formula for calculating, from the ability value, a health age relating to the motor function as the score.

7. The information processing system according to claim 5,
wherein the subject's attribute is defined by an age of a subject, and
wherein the calculation formula is a calculation formula for calculating a health age relating to the motor function as the score, and is configured to calculate the health age such that the health age of the second subject whose ability value is the standard value corresponds to an actual age of that second subject.

8. The information processing system according to claim 6, wherein the subject's attribute is defined by a combination of the age and a sex.

9. The information processing system according to claim 5, wherein
the calculation formula setter is configured to
derive a regression model based on a regression analysis using, as samples, the ability values of the first subjects, the regression model including an explanatory variable corresponding to the subject's attribute and an objective variable corresponding to the ability value, the regression model defining the standard value of the ability values in each attribute class, and
set the calculation formula so as to allocate, based on the standard value of the ability values and on a standard deviation corresponding to a variation from the standard value of the ability values of the first subjects, a range of the ability values that has a width proportional to the standard deviation centered at the standard value to the specific range of the score, in each attribute class.

10. The information processing system according to claim 1, wherein the ability values are values calculated in accordance with an item response model based on an item response theory, the item response model using the ability value of a subject and a difficulty level of a respective one of the test items and thereby representing an occurrence probability relating to each determination value that possibly takes for the respective one of the test items.

11. An information processing method implemented by a computer, the method comprising:
calculating ability values of first subjects based on first test data, each of the ability values being an ability value relating to a motor function of a body of a corresponding one of the first subjects, the first test data describing a test result relating to the motor function of each of the first subjects, the test result including determination values, for respective test items, for a corresponding one of the first subjects;
setting a calculation formula for a score corresponding to an ability value based on a distribution of the ability values calculated relative to a subject's attribute;
calculating the score corresponding to an ability value of a second subject relating to the motor function based on the ability value of the second subject and on the calculation formula, the ability value being calculated based on second test data, the second test data including determination values, for the respective test items relating to the motor function of the second subject; and
outputting the score calculated.

12. The information processing method according to claim 11,
wherein the subject's attribute is defined by an age of a subject, and
wherein the calculation formula is a calculation formula for calculating, from the ability value, a health age relating to the motor function as the score.

13. The information processing method according to claim 12,
wherein setting the calculation formula comprises setting the calculation formula in accordance with a regression model between the age and the ability value, based on a regression analysis using, as samples, the ability values of the first subjects.

14. An information processing method implemented by a computer, the method comprising:
calculating ability values of first subjects based on first test data, each of the ability values being an ability value relating to a motor function of a body of a corresponding one of the first subjects, the first test data describing a test result relating to the motor function of each of the first subjects, the test result including determination values, for respective test items, for a corresponding one of the first subjects;
setting a calculation formula for a score corresponding to an ability value in a respective one of attribute classes included in a subject's attribute, based on a distribution of the ability values calculated in the respective one of the attribute classes;
calculating the score corresponding to an ability value of a second subject relating to the motor function based on the ability value of the second subject and on the calculation formula in one of the attribute classes corresponding to the second subject, the ability value being calculated based on second test data, the second test data including determination values, for the respective test items relating to the motor function of the second subject; and
outputting the score calculated.

15. The information processing method according to claim 14, wherein setting the calculation formula comprises setting the calculation formula in each attribute class based on a standard value of the ability values of a subject group in a corresponding attribute class and on a standard deviation of the ability values of the subject group in the corresponding attribute class such that a specific range of the score is allocated to a range of the ability values that has a width proportional to the standard deviation centered at the standard value.

16. The information processing method according to claim 14,
wherein the subject's attribute is defined by an age of a subject, and
wherein the calculation formula is a calculation formula for calculating, from the ability value, a health age relating to the motor function as the score.

17. The information processing method according to claim 11, wherein the ability values are values calculated in accordance with an item response model based on an item response theory, the item response model using the ability value of a subject and a difficulty level of a respective one of the test items and thereby representing an occurrence probability relating to each determination value that possibly takes for the respective one of the test items.

18. (canceled)

19. The information processing method according to claim 15,
wherein the subject's attribute is defined by an age of a subject, and
wherein the calculation formula is a calculation formula for calculating a health age relating to the motor function as the score, and is configured to calculate the health age such that the health age of the second subject whose ability value is the standard value corresponds to an actual age of that second subject.

20. The information processing method according to claim 16, wherein the subject's attribute is defined by a combination of the age and a sex.

21. The information processing method according to claim 15, wherein
setting the calculation formula comprises
deriving a regression model based on a regression analysis using, as samples, the ability values of the first subjects, the regression model including an explanatory variable corresponding to the subject's attribute and an objective variable corresponding to the ability value, the regression model defining the standard value of the ability values in each attribute class, and
setting the calculation formula so as to allocate, based on the standard value of the ability values and on the standard deviation corresponding to a variation from the standard value of the ability values of the first subjects, a range of the ability values that has a width proportional to the standard deviation centered at the standard value to the specific range of the score, in each attribute class.
